(19) United States
(12) Patent Application Publication
(10) Pub. No.: US 2017/0079538 A1
(43) Pub. Date: Mar. 23, 2017

(54) Method for Identifying Images of Brain Function and System Thereof

(71) Applicant: National Central University, Taoyuan City (TW)

(72) Inventors: Wei-Kuang LIANG, Taoyuan City (TW); Norden E. HUANG, Taoyuan City (TW); Chi-Hung JUAN, Taoyuan City (TW)

(21) Appl. No.: 14/928,906

(22) Filed: Oct. 30, 2015

(30) Foreign Application Priority Data
Sep. 17, 2015 (TW) 104130789

(51) Int. Cl. A61B 5/04; A61B 5/00; A61B 5/0476

(52) U.S. Cl. CPC A61B 5/04012; A61B 5/0476; A61B 5/04008; A61B 5/4064

(57) ABSTRACT

The present invention provides a method for identifying images of brain function. In the beginning, choosing one of the brain data collected by multichannel scalp EEG/MEG, and using a mode decomposition method to obtain a plurality of intrinsic mode functions for each brain data, transforming the intrinsic mode functions (IMFs) in the same frequency scale into a plurality of source IMFs across the cerebral cortex by a source reconstruction algorithm, and classifying each source IMF in the same frequency scale into a plurality of frequency regions corresponding to the different brain sites. Then, repeatedly choosing a source IMF, and obtaining an amplitude envelope line through each absolution value of the source IMF. Further to obtain a plurality of source first-layer amplitude IMFs decomposed from the function of the amplitude envelope line by the mode decomposition method. Until obtaining the source first-layer amplitude IMFs from each source IMF, classifying each source first-layer amplitude IMF in the same amplitude frequency scale into a plurality of amplitude frequency regions corresponding to the different brain sites. In the end, a brain amplitude modulation spectrum is provided for analyzing the relationship between each frequency region and each amplitude frequency region.

### CROSS-REFERENCE TO RELATED APPLICATIONS

This Non-provisional application claims priority under 35 U.S.C. §119(a) on Patent Application No(s). [104130789] filed in Taiwan, Republic of China [Sep. 17, 2015], the entire contents of which are hereby incorporated by reference.

### FIELD OF THE INVENTION

The present invention provides a method and a system for identifying images of brain function. In particular, the method and the system generate a brain amplitude modulation spectrum by Holo-Hilbert Analysis (HHSA) and a source reconstruction method.

### BACKGROUND OF THE INVENTION

Functional 3D brain imaging, such as functional magnetic resonance imaging (fMRI), Near-infrared spectroscopy (NIRS) and Positron Emission Tomography (PET), are useful tools to give a high spatial resolution functional map of the brain. However, fMRI, NIRS and PET has low temporal resolution that put a severe limitation on all these tools for investigating dynamics of neural activities in the brain. Conversely, other non-imaging brain activities measurement techniques such as electroencephalogram (EEG) or magnetoencephalogram (MEG) are useful to give high temporal resolution data to characterize the dynamics of the brain, however EEG and MEG relatively low spatial resolution and limited all to the data from cerebral cortex also limited their usefulness to identify the sources of brain activities originated from places other than the cerebral cortex. Although there are existing efforts to combine source reconstruction techniques and frequency analysis methods (e.g. Band-pass filter, Fast-Fourier Transform, Wavelet Transform) to estimate the 3D oscillatory sources in the brain, and showed great improvement in the area of oscillatory source localization, one common shortcoming among them all is the difficulty rooted on the flawed linear stationary based Fourier type of frequency analysis, which failed to reveal some crucial characteristics of brain signals such as nonlinearity and inter-mode interactions that are known to be able to critically modulate our physical or mental states (e.g. behavioral performance, attention, working memory, aging, and degree of an illness).

### SUMMARY OF THE INVENTION

The present invention provides a method and a system for identifying images of brain function, and more particularly, the method and the system transform the brain signals into 3D (amplitude modulation, frequency modulation and time) spectrum by Holo-Hilbert Analysis, and the plurality of brainwave data is recorded from multiple channels placed on or over the scalp grouped at same time domain, further to quantify the synchronization relationship between different brain regions. Therefore, the present invention provides an amplitude modulation spectrum for early detection of brain diseases and psychological diseases.

In accordance with another embodiment, the method implemented in a data analysis system for identifying images of brain function comprises obtaining a plurality of brainwave data, wherein the plurality of brainwave data is electroencephalography (EEG) or magnetoencephalography (MEG) recorded from multiple channels placed on or over the scalp; selecting one of the brainwave data decomposed by a mode decomposition method to obtain a plurality of intrinsic mode functions (IMFs), wherein the plurality of intrinsic mode functions are an amplitude value changes over time of the brainwave data in each different frequency scale; selecting another one of the brainwave data, repeating the last step, until obtaining the plurality of intrinsic mode functions from all of the brainwave data; then, classifying the plurality of intrinsic mode functions in the same frequency scale into a frequency region, to obtain a plurality of frequency regions corresponding to the different EEG or MEG channels.

Furthermore, obtaining a plurality of source intrinsic mode functions corresponding to the different brain sites based on a source reconstruction method to transform the plurality of intrinsic mode functions in the same frequency scale into a source space, until transforming all of intrinsic mode functions into the source intrinsic mode functions; selecting one of the source intrinsic mode functions, taking an absolute value of the source intrinsic mode function, then producing an amplitude envelope line comprising all maxima of the absolute value, to obtain a plurality of source first-layer amplitude intrinsic mode functions from the amplitude envelope line by the mode decomposition method, wherein the plurality of source first-layer amplitude intrinsic mode functions are a value changes over time of the amplitude envelope line in each different amplitude frequency scale; selecting another one of the source intrinsic mode functions, repeating the last step, until obtaining the plurality of source first-layer amplitude intrinsic mode functions from all of the source intrinsic mode functions; classifying the plurality of source first-layer amplitude intrinsic mode functions in the same amplitude frequency scale into a plurality of amplitude frequency regions corresponding to the different brain sites.

Finally, generating a brain amplitude modulation spectrum based on the plurality of frequency regions corresponding to the plurality of amplitude frequency regions at same time, wherein the brain amplitude modulation spectrum discloses a plurality of relative values between the frequency regions and the amplitude frequency regions corresponding to the different brain sites.

In accordance with another embodiment, a system for identifying images of brain function comprises a signal received unit, a data processing unit, a region selection unit and a signal spectrum combined unit.

The signal received unit obtains a plurality of brainwave data, wherein the plurality of brainwave data is electroencephalography or magnetoencephalography recorded from multiple channels placed on or over the scalp.

The data processing unit is connected with the signal received unit for selecting one of the brainwave data, then decomposing the brainwave data to obtain a plurality of intrinsic mode functions by a mode decomposition method, wherein the plurality of intrinsic mode functions are an amplitude value changes over time of the brainwave data in each different frequency scale, until obtaining the plurality of intrinsic mode functions from all of the brainwave data.

Furthermore, the data processing unit performs a source reconstruction method to transform the plurality of intrinsic mode functions in the same frequency scale into a source space, to obtain a plurality of source intrinsic mode functions corresponding to the different brain sites, then selecting another one of the source intrinsic mode functions, executing the last step repeatedly until obtaining the plurality of source first-layer amplitude intrinsic mode functions from all of the source intrinsic mode functions, and selecting one of the source intrinsic mode functions, taking an absolute value of the source intrinsic mode function to produce an amplitude envelope line comprising all maxima of the absolute value. The data processing unit further performs the mode decomposition method to obtain a plurality of source first-layer amplitude intrinsic mode functions of the amplitude envelope line, wherein the plurality of source first-layer amplitude intrinsic mode functions are a value changes over time of the amplitude envelope line in each different amplitude frequency scale. The data processing unit selects another one of the source intrinsic mode functions and executes the last step repeatedly, until obtaining the plurality of source first-layer amplitude intrinsic mode functions from all of the source intrinsic mode functions.

The region selection unit is connected with the data processing unit for classifying the plurality of intrinsic mode functions in the same frequency scale into a plurality of frequency regions corresponding to the different EEG or MEG channels, and classifies the plurality of source first-layer amplitude intrinsic mode functions in the same amplitude frequency scale into a amplitude frequency region corresponding to the different brain sites.

The signal spectrum combined unit is connected with the region selection unit for generating a brain amplitude modulation spectrum based on the plurality of frequency regions corresponding to the plurality of amplitude frequency regions at same time, wherein the brain amplitude modulation spectrum discloses a relative value between the frequency regions and the amplitude frequency regions corresponding to the different brain sites.

The present invention provides a whole brain amplitude modulation spectrum based on amplitude modulation dimensions and frequency modulation dimensions through non-invasive electroencephalogram or magnetoencephalogram. Wherein the whole brain amplitude modulation spectrum discloses the activity of different brain positions by analyzing relationship between amplitude modulation and frequency modulation. Therefore, the present invention can further provide an amplitude modulation spectrum for early detection of brain diseases and psychological diseases.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a block diagram of a system for identifying images of brain function;

FIG. 2 illustrates the correlations between a power of an amplitude modulation spectrum and K value;

FIG. 3 illustrates one example of a brain amplitude modulation spectrum and an orthographic view;

FIG. 4 illustrates one example of a position amplitude modulation spectrum;

FIG. 5 illustrates another example of a brain amplitude modulation spectrum;

FIG. 6 is a flowchart of a method for identifying images of brain function;

FIG. 7 illustrates one example of a binding visual working memory paradigm.

### DETAILED DESCRIPTION OF THE INVENTION

Summarizing various aspects of the present disclosure, this reference will now be made in detail to the description of the disclosure as illustrated in the drawings. While the disclosure will be described in connection with these drawings, there is no intent to limit it to the embodiments disclosed herein. On the contrary, the intent is to cover all alternatives, modifications and equivalents included within the spirit and scope of the disclosure as defined by the appended claims.

The present invention discloses a method implemented in a data analysis system for identifying images of brain function. The method provides merely an example in the different types of functional arraignments that may be employed to implement the operation in the various components of a system for identifying images of brain function, such as a computer system connected to a scanner, a multiprocessor computing device, and so forth. The execution steps of the present invention may include application specific software which may store in any portion or component of the memory including, such as random access memory (RAM), read-only memory (ROM), hard drive, solid-state drive, magneto optical (MO), IC chip, USB flash drive, memory card, optical disc such as compact disc (CD) or digital versatile disc (DVD), floppy disk, magnetic tape, or other memory components.

For some embodiments, the system comprises a display device, a processing unit, a memory, an input device and a storage medium. The input device used to provide data such as image, text or control signals to an information processing system such as a computer or other information appliance. In accordance with some embodiments, the storage medium such as, by way of example and without limitation, a hard drive, an optical device or a remote database server coupled to a network, and stores software programs. The memory typically is the process in which information is encoded, stored, and retrieved etc. The processing unit performs data calculations, data comparisons, and data copying. The display device is an output device that visually conveys text, graphics, and the brain amplitude modulation spectrum. Information shown on the display device is called soft copy because the information exists electronically and is displayed for a temporary period of time. The display device includes CRT monitors, LCD monitors and displays, gas plasma monitors, and televisions. In accordance with some embodiments, the software programs are stored in the memory and executed by the processing unit when the computer system executes the method for identifying images of brain function. Finally, information provided by the processing unit, and presented on the display device or stored in the storage medium.

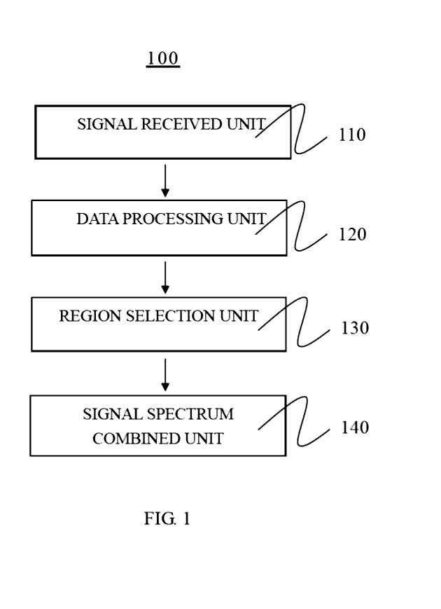

Please refer FIG. 1, FIG. 1 is a block diagram of a system for identifying images of brain function in accordance with some embodiments of the present disclosure. The system 100 for identifying images of brain function comprises a signal received unit 110, a data processing unit 120, a region selection unit 130 and a signal spectrum combined unit 140, wherein the data processing unit 120 is connected with the signal received unit 110, the region selection unit 130 is connected with the data processing unit 120 and the signal spectrum combined unit 140 is connected with the region selection unit 130.

After the signal received unit 110 receives a plurality of brainwave data, the data processing unit 120 will decompose one of the brainwave data, wherein the sampling frequency is over 64 Hz to contain gamma frequency regions. The plurality of brainwave data is electroencephalography or magnetoencephalography recorded from multiple channels placed on or over the scalp.

The data processing unit 120 decomposes the plurality of brainwave data to obtain a plurality of intrinsic mode functions (IMFs) by a mode decomposition method, wherein the plurality of intrinsic mode functions are an amplitude value changes over time of the brainwave data in each different frequency scale, until obtaining the plurality of intrinsic mode functions from all of the brainwave data.

The mode decomposition method may include by way of example and without limitation, such as Empirical Mode Decomposition (EMD), Ensemble Empirical Mode Decomposition (EEMD) and Conjugate Adaptive Dyadic Masking Empirical Mode Decomposition (CADM-EMD). The mode decomposition method decomposes the brainwave data to obtain the plurality of intrinsic mode functions. Beside the mode decomposition method mentions above, the plurality of intrinsic mode functions may include by way of example and without limitation, decomposed by Adaptive Filtering or Optimal Basis Pursue. The region selection unit 130 classifies the plurality of intrinsic mode functions in the same frequency scale into a plurality of frequency regions corresponding to the different EEG or MEG channels.

Furthermore, the data processing unit 120 performs a source reconstruction method, for example, beamformer, minimum norm estimation (MNE), eLORETA or multiple sparse priors and uses a forward model, for example, spherical model, boundary element model (BEM), and finite element model (FEM) on sources over a 2D cortical mesh, 3D cortical mesh or a 3D grid derived from a template (e.g. MNI template) or a 3D structure magnetic resonance imaging (MRI) to transform the plurality of intrinsic mode functions in the same frequency scale into a source space, to obtain a plurality of source intrinsic mode functions corresponding to the different brain sites. The data processing unit 120 selects another one of the intrinsic mode functions, and executes the last step repeatedly until obtaining the plurality of source intrinsic mode functions from all of the intrinsic mode functions.

The data processing unit 120 selects one of the source intrinsic mode functions, and takes an absolute value of the source intrinsic mode function to produce an amplitude envelope line comprising all maxima of the absolute value. The data processing unit 120 further performs the mode decomposition method to obtain a plurality of source first-layer amplitude intrinsic mode functions from the amplitude envelope line, wherein the plurality of source first-layer amplitude intrinsic mode functions are a value changes over time of the amplitude envelope line in each different amplitude frequency scale. The data processing unit 120 selects another one of the source intrinsic mode functions, and executes the last step repeatedly until obtaining the plurality of source first-layer amplitude intrinsic mode functions from all of the source intrinsic mode functions. The region selection unit 130 classifies the plurality of source first-layer amplitude intrinsic mode functions in the same amplitude frequency scale into an amplitude frequency region corresponding to the different brain sites.

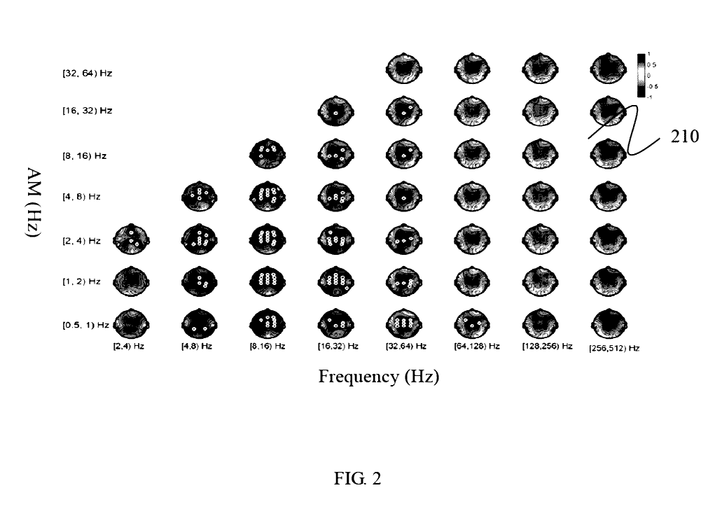

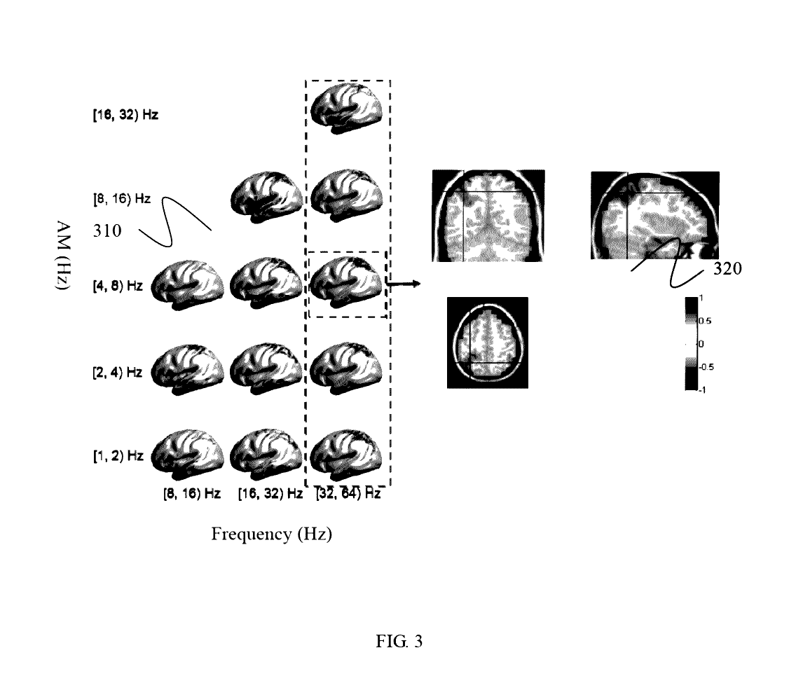

Please refer FIG. 2 and FIG. 3, FIG. 2 illustrates an amplitude modulation spectrum of the correlations between the Holo-Hilbert Spectrum power (marginal sum over each dyadic window of both the amplitude modulation and frequency dimensions) and the K value over all EEG channels, and FIG. 3 illustrates one example of a brain amplitude modulation spectrum and an orthographic view.

The signal spectrum combined unit 140 generates a brain amplitude modulation spectrum 310, for example, a Dynamic EEG Projected Brain Tomographic Image (deepBTGI) based on the plurality of frequency regions corresponding to the plurality of amplitude frequency regions at same time, wherein the brain amplitude modulation spectrum 310 is the relative value of the frequency region and the amplitude frequency region corresponding to the different brain sites. The brain amplitude modulation spectrum is also provided for analyzing the relationship between each frequency region and each amplitude frequency region.

In FIG. 2, each amplitude modulation spectrum illustrates the correlations between the Holo-Hilbert Spectrum power and the K value. The different shades of color in each brain amplitude modulation spectrum denote correlation coefficients, and small white circles denote the correlations on those EEG channels are significant statistically. FIG. 3 illustrates correlations between the power of the brain amplitude modulation spectrum and K value of AM 1-32 Hz over frequency 8-64 Hz. The different shades of color in each tomography denote correlation coefficients, and the results are masked by a statistical result (p<0.01 under a cluster-based nonparametric permutation test). In FIG. 3 further shows an orthographic view 320 providing a dyadic tomography of amplitude modulation (AM) 4-8 Hz over frequency 32-64 Hz.

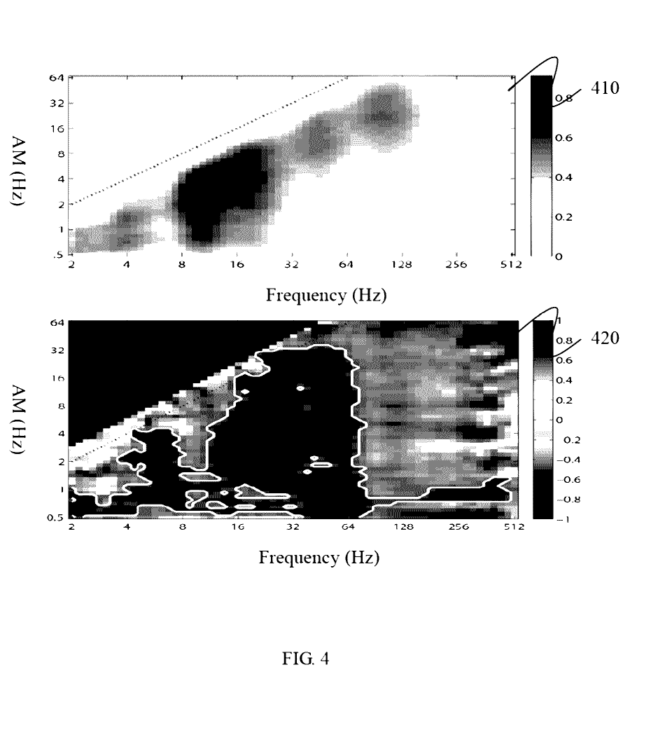

Please refer FIG. 4, FIG. 4 illustrates one example of a position amplitude modulation spectrum. The signal spectrum combined unit 140 selects one of the brain sites, then generates a position amplitude modulation spectrum 410-420 based on the plurality of source intrinsic mode functions corresponding to the plurality of source first-layer amplitude intrinsic mode functions at same time, wherein the position amplitude modulation spectrum 410-420 is the relative value of the plurality of source intrinsic mode functions and the plurality of source first-layer amplitude intrinsic mode functions at the same brain site. The position amplitude modulation spectrum discloses the relationship between the plurality of source intrinsic mode functions and the plurality of source first-layer amplitude intrinsic mode functions. The signal spectrum combined unit 140 selects another one of the brain sites, and executes the last step repeatedly until obtaining the position amplitude modulation spectrums for all of brain sites.

In FIG. 4, the 1st position amplitude modulation spectrum 410 shows the averaged Holo-Hilbert Spectrum (HHS) power during the memory retention interval on left posterior parietal cortex for the “Hit” trials, where participant successfully detected the changes in test array. In FIG. 4, the 2nd position amplitude modulation spectrum 420 shows the correlations between the Holo-Hilbert Spectrum power and the K value, where K value is a behavioral index of working memory capacity. In the 2nd spectrum 420, areas enclosed by white contours denote significant (p<0.05, two-tailed) are under a cluster-based nonparametric permutation test.

For some embodiments, the signal spectrum combined unit 140 compares the position amplitude modulation spectrums, which obtained after the patient memorizes the study array and the test array for determining changes of the relative value between the frequency regions and the amplitude frequency regions corresponding to the different brain sites, wherein the relative value is the relationship between each frequency region and each amplitude frequency region. The signal spectrum combined unit 140 compares the brain amplitude modulation spectrums, which obtained after the patient memorizes the study array and the test array for determining the relative value changes between the plurality of source intrinsic mode functions and the plurality of source first-layer amplitude intrinsic mode functions at the same brain site, wherein the relative value is the relationship between each source intrinsic mode function and each plurality of source first-layer amplitude intrinsic mode function.

For some embodiments, after the signal received unit 110 receives the plurality of brainwave data. The data processing unit 120 decomposes one of the brainwave data, wherein the plurality of brainwave data is collected at no less than the standard 32 different brain sites from the patient, at the sampling frequency no less than 512 Hz. Before the signal received unit 110 receives the plurality of brainwave data, the patient is requested to memorize the study array first, wherein the plurality of brainwave data is electroencephalography or magnetoencephalography recorded from multiple channels placed on or over the scalp.

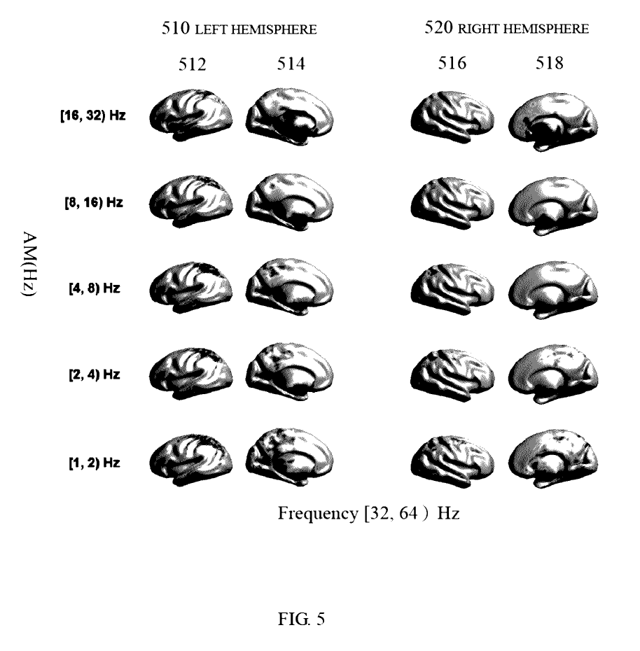

For some embodiments, please refer FIG. 5, FIG. 5 illustrates another example of a brain amplitude modulation spectrum. The signal spectrum combined unit 140 generates the brain amplitude modulation spectrum comprises the memory retention interval on the left lateral and medial, right lateral and medial views (from left to right, respectively) for determining the relative value changes between the frequency regions and the amplitude frequency regions corresponding to the different brain sites. The signal spectrum combined unit 140 further compares the brain amplitude modulation spectrums, which obtained after the patient memorizes the study array, to the brain amplitude modulation spectrums, which obtained after the patient memorizes the test array for determining the relative value changes between the plurality of source intrinsic mode functions and the plurality of source first-layer amplitude intrinsic mode functions at the same brain site.

The method for identifying images of brain function provides the study array for the patient to memorize. After a short retention interval, the patient is required to memorize the test array, and then another brain amplitude modulation spectrum is obtained for indicating any changes between the study array and the test array. In FIG. 5, the left hemisphere 510, the right hemisphere 520, lateral 512-516 and medial 514-518 and medial views (from left to right, respectively) of the brain amplitude modulation spectrum of AM 1-32 Hz over frequency 32-64 Hz. The brain amplitude modulation spectrum shows clear concentration of energy and also amplitude modulations on gamma frequencies in the region approximately at the hippocampus, the region has been recognized as an essential region for maintaining information for both working memory and long-term memory.

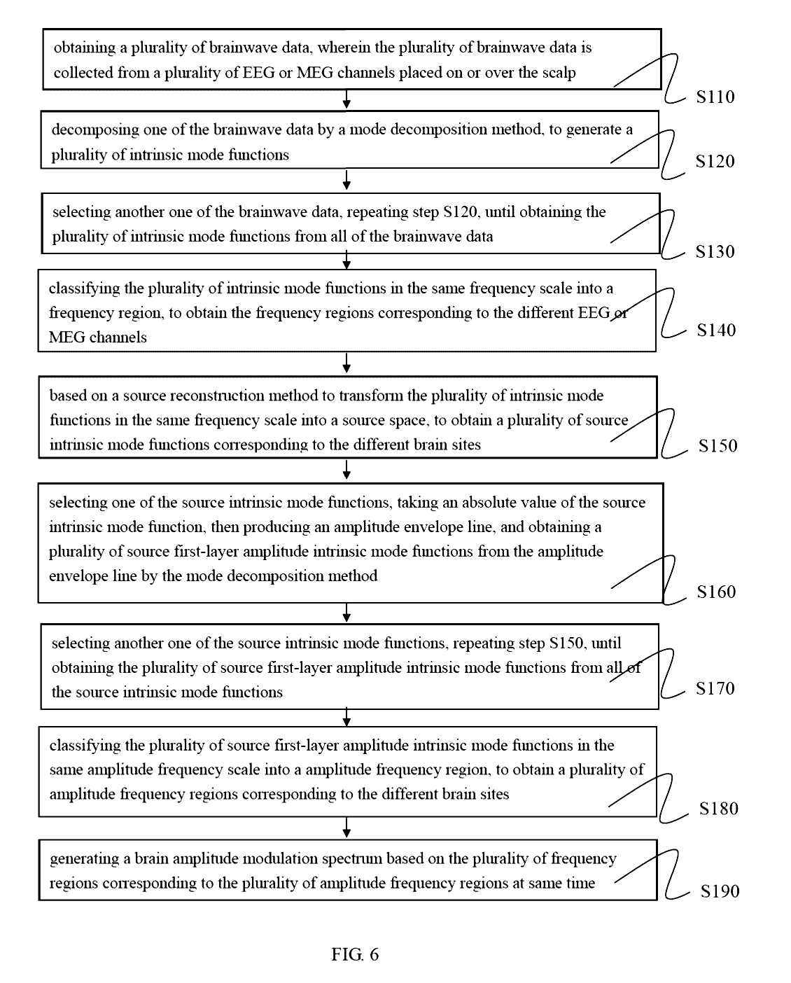

In FIG. 6 is a flowchart that provides one example of a method S100 for identifying images of brain function, according to some embodiments. First of all, in step S110, the signal received unit 110 receives a plurality of brainwave data, wherein the plurality of brainwave data is collected from a plurality of EEG or MEG channels. After the signal received unit 110 receives the plurality of brainwave data, then the data processing unit 120 receives the plurality of brainwave data, and decomposes one of the brainwave data, wherein the sampling frequency is over 64 Hz to contain gamma frequency regions. The plurality of brainwave data is electroencephalography or magnetoencephalography recorded from multiple channels placed on or over the scalp.

In step S120, The data processing unit 120 selects one of brainwave data to obtain a plurality of intrinsic mode functions by a mode decomposition method, wherein the plurality of intrinsic mode functions are an amplitude value changes over time of the brainwave data in each different frequency scale.

In step S130, the data processing unit 120 selects one of the brainwave data, executes the last step repeatedly, until obtaining the plurality of intrinsic mode functions from all of the brainwave data. In an embodiment, the mode decomposition method may include by way of example and without limitation, such as empirical mode decomposition, ensemble empirical mode decomposition and conjugate adaptive dyadic masking empirical mode decomposition. The mode decomposition method decomposes the brainwave data to obtain the plurality of intrinsic mode functions. Beside the mode decomposition method mentions above, the plurality of intrinsic mode functions may include by way of example and without limitation, decomposed by adaptive filtering or optimal basis pursue.

Further, in step S140, the region selection unit 130 classifies the plurality of intrinsic mode functions in the same frequency scale into a frequency region corresponding to the different EEG or MEG channels.

In step S150, the data processing unit 120 performs a source reconstruction method, for example, beamformer, minimum norm estimation (MNE), eLORETA or multiple sparse priors and uses a forward model, for example, spherical model, boundary element model, and finite element model on sources over a 2D cortical mesh, 3D cortical mesh or a 3D grid derived from a template (e.g. MNI template) or a 3D structure magnetic resonance imaging (MRI) to transform the plurality of intrinsic mode functions in the same frequency scale into a source space to obtain a plurality of source intrinsic mode functions corresponding to the different brain sites. Then, the data processing unit 130 selects another one of the source intrinsic mode functions and executes the last step repeatedly, until obtaining the plurality of source first-layer amplitude intrinsic mode functions from all of the source intrinsic mode functions.

In step S160, the data processing unit 120 selects one of the source intrinsic mode functions, and takes an absolute value of the source intrinsic mode function to produce an amplitude envelope line comprising all maxima of the absolute value. The data processing unit 120 further performs the mode decomposition method to obtain the plurality of source first-layer amplitude intrinsic mode functions from the amplitude envelope line.

In step S170, the data processing unit 120 selects another one of the source intrinsic mode functions, and executes the step S160 repeatedly, until obtaining the plurality of source first-layer amplitude intrinsic mode functions from all of the source intrinsic mode functions, wherein the plurality of source first-layer amplitude intrinsic mode functions are a value changes over time of the amplitude envelope line in each different amplitude frequency scale.

Then, in step S180, the region selection unit 130 classifies the plurality of source first-layer amplitude intrinsic mode functions in the same amplitude frequency scale into an amplitude frequency region corresponding to the different brain sites.

Finally, in step S190, the signal spectrum combined unit 140 generates a brain amplitude modulation spectrum 310, for example, a Dynamic EEG Projected Brain Tomographic Image based on the plurality of frequency regions corresponding to the plurality of amplitude frequency regions at same time, wherein the brain amplitude modulation spectrum 310 is the relative value of the frequency region and the amplitude frequency region corresponding to the different brain sites. The brain amplitude modulation spectrum 310 is provided for analyzing the relationship between each frequency region and each amplitude frequency region.

For some embodiments, the signal spectrum combined unit 140 selects one of the brain sites, then generates a position amplitude modulation spectrum based on the plurality of source intrinsic mode functions corresponding to the plurality of source first-layer amplitude intrinsic mode functions at same time, wherein the position amplitude modulation spectrum is the relative value of the plurality of source intrinsic mode functions and the plurality of source first-layer amplitude intrinsic mode functions at the same brain site. The brain amplitude modulation spectrum is provided for analyzing the relationship between the plurality of source intrinsic mode functions and the plurality of source first-layer amplitude intrinsic mode functions. The signal spectrum combined unit 140 selects another one of the brain sites, and executes the last step repeatedly until obtaining the position amplitude modulation spectrums for all of brain sites.

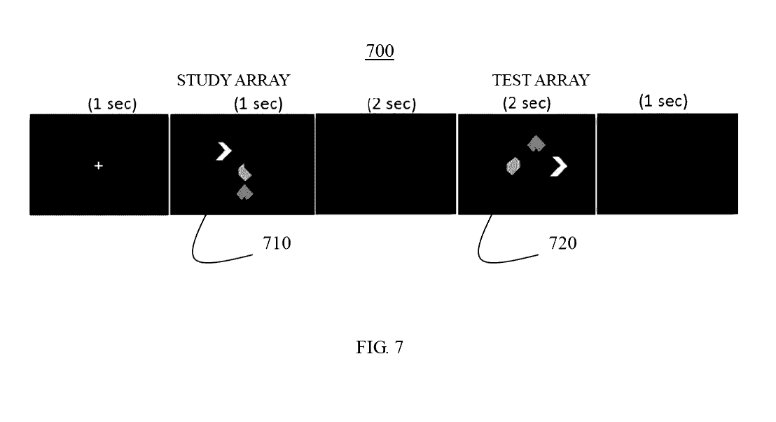

For some embodiments, please refer FIG. 7. FIG. 7 illustrates one example of a Binding Visual Working Memory Paradigm, according to some embodiments. The method for identifying images of brain function provides the study array 710 of the Binding Visual Working Memory Paradigm 700 for the patient to memorize, after a short retention interval, the patient is required to indicate any changes between the study array 710 and the test array 720. In the color-shape binding visual working memory task, the user needs to judge whether the correspondence between both shape and color has changes.

In accordance with some embodiments, a method is implemented in a data analysis system for identifying images of brain function. The method comprises obtaining the plurality of brainwave data, after the patient memorizes the study array 710. Then, repeating step S110 to step S190, after the patient memorize the test array 720. The method comprises selecting one of the brainwave data based on performing the mode decomposition method to obtain the plurality of intrinsic mode functions, until obtaining the plurality of intrinsic mode functions from all of the brainwave data, wherein the plurality of intrinsic mode functions are the amplitude value changes over time of the brainwave data in each different frequency scale, and classifies the plurality of intrinsic mode functions in the same frequency scale into a frequency region corresponding to the different EEG or MEG channels.

Furthermore, obtaining a plurality of source intrinsic mode functions corresponding to the different brain sites based on a source reconstruction method to transform the plurality of intrinsic mode functions in the same frequency scale into a source space, and transform all of intrinsic mode functions into the source intrinsic mode functions. Then, selecting one of the source intrinsic mode functions, taking an absolute value of the source intrinsic mode function, then producing an amplitude envelope line comprising all maxima of the absolute value, and obtaining a plurality of source first-layer amplitude intrinsic mode functions from the amplitude envelope line based on performing the mode decomposition method, until obtaining the plurality of source first-layer amplitude intrinsic mode functions from all of the source intrinsic mode functions, wherein the plurality of source first-layer amplitude intrinsic mode functions are a value changes over time of the amplitude envelope line in each different amplitude frequency scale, and classifying the plurality of source first-layer amplitude intrinsic mode functions in the same amplitude frequency scale into an amplitude frequency region corresponding to the different brain sites.

Finally, generating a brain amplitude modulation spectrum based on the plurality of frequency regions corresponding to the plurality of amplitude frequency regions at same time, wherein the brain amplitude modulation spectrum is obtained, after the patient memorizes the test array.

For some embodiments, the signal spectrum combined unit 140 compares the position amplitude modulation spectrums, which obtained after the patient memorizes the study array with the position amplitude modulation spectrums, which obtained after the patient memorizes the test array for determining the relative value changes between the frequency regions and the amplitude frequency regions corresponding to the different brain sites, wherein the relative value is the relationship between each frequency region and each amplitude frequency region. The signal spectrum combined unit 140 compares the brain amplitude modulation spectrums, which obtained after the patient memorizes the study array and the test array for determining the relative value changes between the plurality of source intrinsic mode functions and the plurality of source first-layer amplitude intrinsic mode functions at the same brain site, wherein the relative value is the relationship between each source intrinsic mode function and each source first-layer amplitude intrinsic mode function.

The present invention provides a method and a system for identifying images of brain function to transform 2D EEG/MEG brain signals into a 3D (spatial coordinates, X, Y, Z)and 3D (Amplitude Modulation , Frequency Modulation and Time) or 3D and 2D (when taking marginal sum over the time dimension within an interval) brain image, the brain image reveals the positions of brain activities dynamically with all full intrinsic functionalities.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed is:

1. A method implemented in a data analysis system for identifying images of brain function, comprises:
(A) obtaining a plurality of brainwave data, wherein the plurality of brainwave data is collected from a plurality of EEG or MEG channels placed on or over the scalp;
(B) decomposing one of the brainwave data by a mode decomposition method, to generate a plurality of intrinsic mode functions, wherein the plurality of intrinsic mode functions are an amplitude value changes over time of the brainwave data in each different frequency scale;
(C) selecting another one of the brainwave data, repeating step (B), until obtaining the plurality of intrinsic mode functions from all of the brainwave data;
(D) classifying the plurality of intrinsic mode functions in the same frequency scale into a frequency region, to obtain a plurality of frequency regions corresponding to the different EEG or MEG channels;
(E) transforming the plurality of intrinsic mode functions in the same frequency scale into a source space by a source reconstruction method, to obtain a plurality of source intrinsic mode functions corresponding to the different brain sites;
(F) selecting one of the source intrinsic mode functions, taking an absolute value of the source intrinsic mode function, then producing an amplitude envelope line comprising all maxima of the absolute value, to obtain a plurality of source first-layer amplitude intrinsic mode functions from the amplitude envelope line by the mode decomposition method, wherein the plurality of source first-layer amplitude intrinsic mode functions are a value changes over time of the amplitude envelope line in each different amplitude frequency scale;
(G) selecting another one of the source intrinsic mode functions, repeating step (F), until obtaining the plurality of source first-layer amplitude intrinsic mode functions from all of the source intrinsic mode functions;
(H) classifying the plurality of source first-layer amplitude intrinsic mode functions in the same amplitude frequency scale into a amplitude frequency region, to obtain a plurality of amplitude frequency regions corresponding to the different amplitude frequency scales; and
(I) generating a brain amplitude modulation spectrum based on the plurality of frequency regions corresponding to the plurality of amplitude frequency regions at same time, wherein the brain amplitude modulation spectrum discloses a plurality of relative values between the frequency regions and the amplitude frequency regions corresponding to the different brain sites.

2. The method of claim 1, the steps further comprises:
(F1) selecting one of the brain sites, and generating a position amplitude modulation spectrum based on the plurality of source intrinsic mode functions corresponding to the plurality of source first-layer amplitude intrinsic mode functions at same time, wherein the position amplitude modulation spectrum discloses a plurality of relative values between the source intrinsic mode functions and the source first-layer amplitude intrinsic mode functions at the same brain site; and
(F2) selecting another one of the brain sites, repeating step (F1), until obtaining the plurality of position amplitude modulation spectrums for all of brain sites.

3. The method of claim 1, wherein in step (A), a patient memorizes a study array first when obtaining the plurality of brainwave data.

4. The method of claim 3, the steps further comprising:
(J) the patient memorizes a test array first, and repeating step (A) to (I), to obtain another brain amplitude modulation spectrum;
(K) comparing the position amplitude modulation spectrum after the patient memorizes the study array to the position amplitude modulation spectrum after the patient memorizes the test array, and determining the relative value changes between the frequency regions and the amplitude frequency regions corresponding to the different brain sites; and
(L) comparing the brain amplitude modulation spectrum after the patient memorizes the study array to the brain amplitude modulation spectrum after the patient memorizes the test array ,and determining the relative value changes between the source intrinsic mode functions and the source first-layer amplitude intrinsic mode functions at the same brain site.

5. The method of claim 1, wherein the plurality of brainwave data is electroencephalography(EEG) or magnetoencephalography(MEG) recorded from multiple channels placed on or over the scalp.

6. The method of claim 1, wherein the mode decomposition method comprises empirical mode decomposition, ensemble empirical mode decomposition or conjugate adaptive dyadic masking empirical mode decomposition.

7. The method of claim 1, wherein the source reconstruction method comprises beamformer, minimum norm estimation, eLORETA or multiple sparse priors.

8. The method of claim 1, wherein the source space is obtained by using a spherical model, a boundary element model or a finite element model over a 2D cortical mesh or a 3D cortical mesh.

9. The method of claim 1, wherein the source space is a template or a 3D structure formed by magnetic resonance imaging.

10. The method of claim 1, the plurality of brainwave data are collected by random or following a regular pattern from one part of EEG or MEG channels placed on or over the scalp.

11. The method of claim 10, the steps further comprises:
(H) repeating to obtain the plurality of brainwave data from another part of EEG or MEG channels, and implementing step (A) to (I), to obtain the plurality of brain amplitude modulation spectrums, then calculating the brain amplitude modulation spectrums by an ensemble average, to obtain an ensemble brain amplitude modulation spectrum.

12. A system for identifying images of brain function, comprises:
a signal received unit, to obtain a plurality of brainwave data, wherein the plurality of brainwave data is collected from a plurality of EEG or MEG channels placed on or over the scalp;
a data processing unit connected with the signal received unit, to decompose one of the brainwave data by a mode decomposition method, to generate a plurality of intrinsic mode functions, wherein the plurality of intrinsic mode functions are an amplitude value changes over time of the brainwave data in each different frequency scale, until obtaining the plurality of intrinsic mode functions from all of the brainwave data, then based on a source reconstruction method to transform the plurality of intrinsic mode functions in the same frequency scale into a source space, to obtain a plurality of source intrinsic mode functions corresponding to the different brain sites, and selecting one of the source intrinsic mode functions, taking an absolute value of the source intrinsic mode function, then producing an amplitude envelope line comprising all maxima of the absolute value, to obtain a plurality of source first-layer amplitude intrinsic mode functions from the amplitude envelope line by the mode decomposition method, until obtaining the plurality of source first-layer amplitude intrinsic mode functions from all of the source intrinsic mode functions, wherein the plurality of source first-layer amplitude intrinsic mode functions are a value changes over time of the amplitude envelope line in each different amplitude frequency scale;
a region selection unit connected with the data processing unit, to classify the plurality of intrinsic mode functions in the same frequency scale into a frequency region corresponding to the different EEG or MEG channels, and classifying the plurality of source first-layer amplitude intrinsic mode functions in the same amplitude frequency scale into a amplitude frequency region corresponding to the different brain sites; and
a signal spectrum combined unit connected with the region selection unit, to generate a brain amplitude modulation spectrum based on the plurality of frequency regions corresponding to the plurality of amplitude frequency regions at same time, wherein the brain amplitude modulation spectrum is a relative value between the frequency regions and the amplitude frequency regions corresponding to the different brain sites.
